(19) United States
(12) Patent Application Publication
(10) Pub. No.: US 2002/0018799 A1
(43) Pub. Date: Feb. 14, 2002

(54) Collagen tubes for nerve regeneration

(76) Inventors: Myron Spector, Brookline, MA (US); Lothar Schloesser, Darmstadt (DE); Peter Geistlich, Stansstad (CH)

Correspondence Address: ROTHWELL, FIGG, ERNST & MANBECK, P.C., 555 13TH STREET, N.W., SUITE 701, EAST TOWER, WASHINGTON, DC, 20004, US

(21) Appl. No.: 09/885,537

(22) Filed: Jun. 21, 2001

Related U.S. Application Data
(60) Provisional application No. 60/214,848, filed on Jun. 28, 2000.

(51) Int. Cl. A61K 45/00; C12N 5/08

(52) U.S. Cl. 424/423; 424/93.7; 435/368

(57) ABSTRACT

Injured nerves are reconnected and regenerated by inserting injured nerve ends into a collagen tube having an outer compact smooth barrier surface preventing ingrowth of connective tissue, avoiding formation of scar tissue and allowing for unimpaired healing of injured nerves. The tube has an inner fibrous surface opposite the outer smooth barrier surface. The soft fibrous inner surface of the tube facilitates nerve growth promotion.

This application claims the benefit of provisional application Ser. No. 60/214,848, filed Jun. 28, 2000.

### BACKGROUND OF THE INVENTION

1. Field of the Invention

The present invention relates to the field of nerve regeneration.

2. Description of the Background Art.

It is known that injured nerves sometimes can be reconnected by entubulation methods wherein nerve ends are inserted into a silicone tube, which may contain a porous, resorbable collagen-graft-glycosaminoglycan (collagen-GAG or CG) copolymer. Although this method has been utilized to reconnect nerves, use of non-resorbable silicone tubes require a later surgical procedure to remove the tubes.

To avoid the second surgical procedure for removing silicone tubes, resorbable tubes formed of Type I bovine tendon collagen have been utilized. Type I tendon collagen tubes have been formed with sidewall pores of approximately 22 nm (termed “porous collagen”) and sidewall pore diameters of less than 3.8 nm (sometimes incorrectly referred to as “non-porous collagen”). These tubes formed of Type I tendon collagen are formed by applying a viscus gel of the purified Type I collagen fibers onto a rotating mandrel and compressing the material to form closely packed fibers. The tubes are chemically crosslinked and lyophilized. One disadvantage of utilizing tubes formed as described above from Type I tendon collagen is that connective tissue and fibroblasts can penetrate the pores in the Type I tendon collagen tube walls, which leads to formation of scar tissue and impedes reconnection of nerve ends. Additionally, the inner surface of Type I tendon collagen tubes formed as described above may also impede reconnection of nerve ends.

There remains a need in the art for improved methods and structures for regenerating and reconnecting injured nerves.

### SUMMARY OF THE INVENTION

In accordance with the present invention, a nerve regeneration tube with a resorbable sidewall is comprised of collagen material having a compact, smooth outer barrier surface so as to inhibit cell adhesion thereon and act as a barrier to prevent passage of cells therethrough. The tube has a soft fibrous inner surface opposite the smooth barrier surface.

### BRIEF DESCRIPTION OF THE DRAWINGS

FIG. 1 is a schematic side elevational view of a membrane for forming a tube in accordance with one embodiment of the present invention.

FIG. 2 is a schematic end elevational view of a filled tube in accordance with one embodiment of the invention.

FIG. 3 is a side elevational view, partly schematic, of a tube in accordance with one embodiment of the invention.

FIG. 4 is a schematic end elevational view of an overlapped tube in accordance with another embodiment of the invention.

### DETAILED DESCRIPTION OF THE INVENTION

The present invention provides a method and structure for reconnecting and regenerating injured nerves, for example, peripheral spine nerves. The present invention utilizes tubes formed of resorbable collagen material having a compact, smooth outer barrier surface for preventing ingrowth of connective tissue, avoiding formation of scar tissue and allowing unimpaired healing of injured nerves.

The outer barrier surface of a tube in accordance with the present invention inhibits cell adhesion thereon and acts as a barrier to prevent passage of cells therethrough, such as fibroblasts.

The sidewall of an inventive tube in accordance with the present invention has a soft fibrous inner surface opposite the outer smooth barrier surface.

In preferred embodiments of the invention, the inventive tube is a mixture of Type III collagen and Type I collagen, e.g., having a Type III collagen content of about 110% by weight, and a Type I collagen content of about 90-99% by weight. In particularly preferred embodiments, the inventive tube has a Type III collagen content of about 1-5% by weight and a Type I collagen content of about 95-99% by weight.

In preferred embodiments, the sidewall of a tube in accordance with the present invention is derived from collagen membrane tissue from a bovine, porcine or other animal source.

In preferred embodiments, the membrane tissue is peritoneal membrane tissue from young calves.

One suitable material for forming tubes according to the invention is Bio-Gide®, from Ed. Geistlich Söhne AG fir Chemishe Industrie, the assignee of the present invention. The Bio-Gide® material and formation thereof is described in U.S. Pat. No. 5,837,278, incorporated herein by reference.

The Bio-Gide® material contains about 1-5% Type III collagen and about 95-99% Type I collagen.

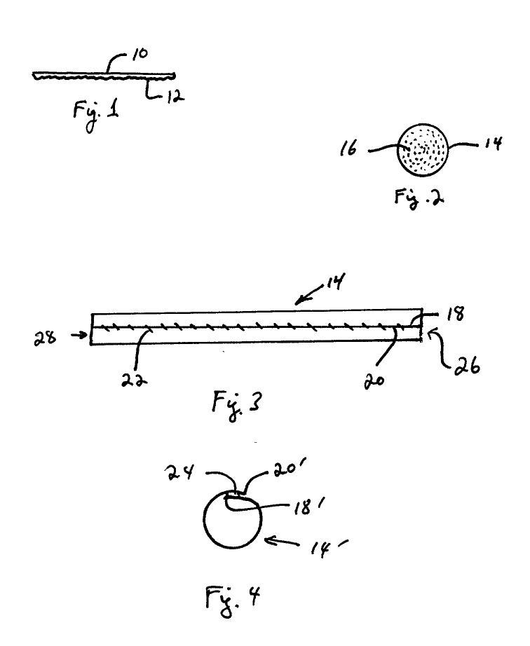

FIG. 1 shows a sheet of collagen material for forming a tube in accordance with the present invention, having a compact, smooth outer barrier surface 10 and a soft fibrous surface 12 opposite the smooth barrier surface 10.

It is believed that the soft fibrous inner surface 12 within a nerve regeneration tube in accordance with the present invention facilitates nerve regeneration.

Nerve regeneration also can be facilitated by providing a nerve growth-promoting filling material within a nerve regeneration tube in accordance with the present invention. In preferred embodiments, the nerve growth-promoting filling material is comprised of Type I collagen, Type IV collagen, or a mixture thereof. Most preferably, the filling material is comprised of collagen fibers having a substantially longitudinal orientation with respect to the axis of the tube. FIG. 2 shows an end-on view of a tube 14 in accordance with the present invention, containing a filling material 16 comprised of collagen fibers having a substantially longitudinal orientation with respect to tube 14.

In particularly preferred embodiments, the filling material 16 is a mixture of Type I collagen and Type IV collagen, most preferably in a ratio of about 1:1 by weight.

The filling material 16 may further contain other ingredients for promoting nerve growth, such as nerve growth stimulants (e.g., laminin), nerve growth factor (NGF), or the like, or mixtures thereof.

In accordance with one embodiment, a nerve regeneration tube in accordance with the present invention is manufactured in a method wherein a sheet of collagen material as described above, such as Bio-Gide®, is provided, and such sheet is formed into a tube. In one embodiment, two opposite side edges 18 and 20 of the sheet of material are brought together to form the tube 14 as shown in FIG. 3. The two opposite side edges 18 and 20 can be joined together by any suitable method to form the tube, such as by utilizing resorbable sutures 22 as shown in FIG. 3, formed of biodegradable threads, e.g., comprised of collagen, polylactid, polyglycolide, or the like. Alternatively, a medically acceptable adhesive may be utilized, such as fibrin glue, starch or collagen slurry.

Referring back to FIG. 2, the nerve growth-promoting filling material 16 may be injected into the tube 14 after formation of tube 14.

Alternatively, the nerve growth-promoting filling material may be formed and freeze-dried to form a collagen sponge, cut into a round cylinder having approximately the diameter of the inner diameter of the tube 14. The sponge cylinder can then be compressed and introduced into the tube after formation of the tube 14.

In still another embodiment, a slurry of the nerve growth-promoting filling material can be applied to the fibrous surface 12 of a sheet of collagen material as shown in FIG. 1 prior to formation of the tube. The tube then can be formed by rolling the membrane sheet with the slurry of filling material attached to the fibrous surface, so as to form the tube with the filling therein in one step. The two side edges can be joined together by sutures, adhesive or the slurry of filling material may act as adhesive.

In the embodiment shown in FIG. 4, the two opposite side edges 18′ and 20′ are overlapped to form tube 14′. The overlap edges 18′ and 20′ can be joined together by sutures or adhesive 24 as shown in FIG. 4. Alternatively, the nerve growth-promoting material may serve as adhesive to join the opposite side edges and form the tube.

When the nerve growth-promoting filling material is provided as a slurry for the tube filling, the filled tubes are freeze-dried for storage prior to use in surgery.

As an alternative to forming the inventive tubes directly from a membrane material such as Bio-Gide®, the tubing sidewall in accordance with the present invention can be made from a collagen slurry so as to provide a compact, smooth outer barrier surface and a fibrous inner surface opposite the smooth barrier surface as described above. The material then can be freeze-dried to form tubes in accordance with the present invention. During use, nerve ends are inserted into the open ends 26 and 28 of a tube 14 in accordance with the present invention to facilitate reconnection of the nerve ends.

The invention is illustrated by the following examples, which are not intended to be limiting.

### EXAMPLE 1

Tubes are formed from Bio-Gide® membranes, with an internal diameter of about 0.5-5 mm and a length of about 10-100 mm. The edges of the tubes are joined by suturing or adhesive.

### EXAMPLE 2

A gel-like Type I collagen mass is produced from porcine rinds as follows. Porcine rinds are minced to a maximum 1 cm size pieces. Water is removed from the porcine rinds with a water-soluble organic solvent, and the solvent is allowed to evaporate. The dried rind pieces are defatted with liquid hydrocarbon solvent. The liquid hydrocarbon solvent is removed, and the dry pieces of rind are allowed to take up water. The hydrated rind pieces are treated with 1 N sodium hydroxide and washed. The pieces of rind are treated with 0.04 N hydrochloric acid solution and washed again. The thus-treated material is ground in a colloid mill to a homogenized liquid slurry containing about 1.5% collagen. The slurry is placed into an injection syringe and tubes formed in accordance with Example 1 are filled with the slurry. The filled tubes are frozen for 24 hours at −20° C. and freeze-dried for 72 hours at a pressure of less than 1 mbar.

### EXAMPLE 3

A filling material comprised of 50% Type I collagen and 50% Type IV collagen is prepared as follows. A 1.5% Type I collagen slurry is prepared from porcine rinds as described in Example 2. Commercially available Type IV collagen is mixed with water in a blender to a 1.5% slurry. The Type I collagen and Type IV collagen slurries are mixed together in the same quantities. The mixed slurries are placed into an injection syringe, and tubes as formed in accordance with Example 1 are filled with the slurry mixture. The tubes are frozen for 24 hours at −20° C., and freeze-dried for 72 hours at a pressure of less than 1 mbar.

### EXAMPLE 4

A slurry in accordance with Example 2 or a mixed slurry in accordance with Example 3 is applied to the fibrous side of Bio-Gide® sheets, and the sheets are rolled to overlap the side edges of the sheets and enclose the slurry while connecting and joining the side edges in one step. The thus-filled tubes then are frozen for 24 hours at −20° C., and freeze-dried for 72 hours at a pressure of less than 1 mbar.

1. A nerve regeneration tube with a resorbable sidewall comprised of collagen material, the sidewall having a compact, smooth outer barrier surface so as to inhibit cell adhesion thereon and act as a barrier to prevent passage of cells therethrough, the sidewall of the tube further having a fibrous inner surface opposite the smooth barrier surface.

2. The tube of claim 1, wherein said sidewall is comprised of a mixture of Type III and Type I collagen.

3. The tube of claim 2, wherein said mixture contains about 1-10% Type III collagen and about 90-99% Type I collagen.

4. The tube of claim 3, wherein said mixture contains about 1-5% Type III collagen and about 95-99% Type I collagen.

5. The tube of claim 1, containing a filling material comprised of Type I collagen, Type IV collagen, or a mixture thereof.

6. The tube of claim 5, wherein the filling material is comprised of collagen fibers having a substantially longitudinal orientation with respect to said tube.

7. The tube of claim 5, wherein said filling material is a mixture of Type I collagen and Type IV collagen.

8. The tube of claim 7, wherein the Type I collagen and the Type IV collagen of said filling material is in a ratio of about 1:1 by weight.

9. The tube of claim 5, wherein said filling material further includes a nerve growth stimulant, nerve growth factor or a mixture thereof.

10. The tube of claim 9, wherein said filling material contains laminin as a nerve growth stimulate.

11. The tube of claim 1, wherein said sidewall is derived from collagen membrane tissue.

12. The tube of claim 11, wherein said membrane tissue is peritoneal tissue.

13. A nerve regeneration tube with a sidewall comprising collagen material derived from collagen membrane tissue.

14. The tube of claim 13, wherein said collagen membrane tissue is peritoneal membrane tissue.

15. A method of producing a nerve regeneration tube as claimed in claim 1, comprising:
a) providing a sheet of collagen material having a compact, smooth outer barrier surface so as to inhibit cell adhesion thereon and act as a barrier to prevent passage of cells therethrough, and a fibrous surface opposite the smooth barrier surface; and
b) forming said sheet into a tube having a sidewall with said compact, smooth outer barrier surface oriented outwardly, said sidewall having an inner surface comprised of said fibrous surface opposite said smooth barrier surface.

16. The method of claim 15, wherein said sheet of collagen material has two opposite side edges, and the two side edges of said sheet are brought together to form said tube from said sheet.

17. The method of claim 16, further including a step of joining said two side edges together to form said tube from said sheet.

18. The method of claim 17, wherein the two side edges are joined together by sutures or adhesive.

19. The method of claim 15, wherein said sheet is formed into said tube with a filling material in said tube comprised of Type I collagen, Type IV collagen or a mixture thereof.

20. The method of claim 15, wherein said sheet has two opposite sides which are overlapped to form said tube.

21. The method of claim 20, wherein said sheet is formed into said tube with a filling material in said tube comprised of Type I collagen, Type IV collagen or a mixture thereof.
